(19) United States
(12) Patent Application Publication
(10) Pub. No.: US 2023/0245757 A1
(43) Pub. Date: Aug. 3, 2023

(54) SYSTEM AND METHOD FOR PROVIDING A FOOD RECOMMENDATION BASED ON FOOD SENSITIVITY TESTING

(71) Applicant: Biomerica, Inc., Irvine, CA (US)

(72) Inventors: Zackary Irani-Cohen, Irvine, CA (US); Elisabeth Laderman, Irvine, CA (US)

(21) Appl. No.: 18/101,912

(22) Filed: Jan. 26, 2023

Related U.S. Application Data
(63) Continuation of application No. 15/875,900, filed on Jan. 19, 2018.
Application No. 15/875,900 is a continuation of application No. PCT/US2016/043688, filed on Jul. 22, 2016.
(60) Provisional application No. 62/195,663, filed on Jul. 22, 2015.

(51) Int. Cl. G16H 20/60; G06Q 50/12; G06Q 20/20; G16H 50/30; G06Q 30/0601; G09B 5/02; G09B 19/00

(52) U.S. Cl. CPC G16H 20/60; G06Q 50/12; G06Q 20/20; G16H 50/30; G06Q 30/0631; G09B 5/02; G09B 19/0092; G16H 50/20

(57) ABSTRACT

Systems, methods, and computer-readable non-transitory storage medium for protecting a patient from adverse reaction to a food ingredient are provided. The system derives a first confidence level data indicating a probability of the patient having adverse reaction to the food ingredient from the patient's medical data. The system obtains food ingredient information comprising a second confidence level data indicating a probability of the food ingredient existing in the food item. Based on the first and second confidence level data, the system generates a safety level for the patient to consume the food item. Then, the system can cause a machine to take an action associated with the food item according to the generated safety level or display a recommendation to the patient.

This application is a continuation of U.S. patent application Ser. No. 15/875,900, filed Jan. 19, 2018, which is a continuation of International Patent Application No. PCT/US2016/043688, filed Jul. 22, 2016, which claims priority to U.S. provisional patent application No. 62/195,663, filed Jul. 22, 2015, which are incorporated by reference herein in their entirety.

### FIELD

The present disclosure relates to methods and systems for providing food and dietary recommendations, and methods and systems for accessing food items based on an individual's sensitivity to specific food or foods.

### BACKGROUND

The following description includes information that may be useful in understanding the present disclosure. It is not an admission that any of the information provided herein is prior art or relevant to the presently claimed invention, or that any publication specifically or implicitly referenced is prior art.

Food sensitivities may result in the display of many different types of symptoms and sicknesses associated with one or more food groups or ingredients. These symptoms or adverse reactions may arise for a wide variety of reasons, making them complex and oftentimes difficult to treat. The cause-effect relationship between food triggers and the resulting symptoms/adverse reactions is not well known, and has not been extensively studied in the medical community. Moreover, meaningful diagnostic methods for determining food sensitivities with respect to some food types have not been well established. With the diagnostics tests that are available, the quality of the test results is generally poor. Typically, problems associated with these tests—as well as the labs interpreting the test results—include high false positive rates, high intra-patient variability, and inter-laboratory variability.

Accurately identifying the various food ingredients present in food items presents another problem. Ambiguous and inconsistent naming conventions amongst food ingredients, and between different food items, can present confusion to the consumer. For example, if a patient does not know with any degree of certainty that a specific food item contains an ingredient that is a trigger for food sensitivity, detection of the ingredients from a food label becomes moot. Similarly, if the food product description on the packaging is ambiguous (e.g., “crab product” versus ‘soft shell crab’, ‘crab imitation meat’, ‘lobster’, etc.), false positive and false negative alerts to the patient may be generated.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there is still a need for improved systems and methods for providing food recommendations or improved food access systems and methods based on a patient's food sensitivity testing and available product information.

### SUMMARY

The subject matter described herein provides systems, methods, and computer-readable non-transitory storage medium for protecting a patient from adverse reaction to a food ingredient.

One aspect of the disclosed subject matter includes a system for protecting a patient from adverse reaction to a food ingredient, which system is communicatively coupled with a machine. The system includes a medical database storing a patient's medical data, and a processor and a memory storing program instructions. The program instructions, when executed by the processor, cause the processor to derive a first confidence level data indicating a probability of the patient having adverse reaction to the food ingredient from the patient's medical data. In some embodiments, the patient's medical data includes patient's food sensitivity data.

In certain embodiments, the program instructions also cause the processor to obtain food ingredient information. In some embodiments, the food ingredient information can be obtained from sensor data representing a food item from a sensor device. Once the sensor data is obtained, the program instructions also cause the processor to derive food ingredient information comprising a second confidence level data indicating a probability of the food ingredient existing in the food item based on the sensor data. Then, the program instructions cause the processor to generate a safety level for the patient to consume the food item based on the first and second confidence level data, and then cause the machine to restrict access of the food item according to the generated safety level.

In some embodiments, the program further causes the processor to perform a step of deriving first confidence level data from data derived from the testing of the patient for a disease using a food preparation having a reference value. In certain embodiments, the reference value comprises an average discriminatory p-value of equal or less than 0.15 for a group of individuals not diagnosed with or suspected of having the disease.

In other embodiments, the program further causes the processor to perform a step of deriving first confidence level data from patient group data of individuals diagnosed of same disease with patient. In certain embodiments, the reference value comprises an average discriminatory p-value of equal or less than 0.15 for a group of individuals not diagnosed with or suspected of having the disease.

In certain embodiments, the reference value in the patient group data is either disease-state stratified or gender stratified to provide more accurate and customized information. Further, the patient group data can include experience data of the individuals diagnosed of same disease.

In addition to the medical data, the program can further causes the processor to derive the first and/or second confidence levels from the patient's experience history and/or group data including others' experience history or sensitivity ratings associated with the food preparations.

In one embodiment, the group data can be updated and self-learned. In this embodiment, the program can further cause the processor to identify a pattern of the group data (e.g., data of the patient group and/or data of the group of individuals not diagnosed of the disease, etc.) correlate the pattern with a probability of the patient having adverse reaction to the food ingredient or with a probability of the food ingredient existing in the food item. Then, the first confidence level and/or the second confidence level can be automatically updated based on patterns of the group data.

The sensor data can be spectral analysis data, chemosensing data, or any other suitable type of data that can provide information regarding the food ingredients. Based on those sensor data, the program can causes the processor to derive the second confidence level by 1) identifying a food ingredient that is likely to exist in the food item and 2) assigning a probability of the food ingredient.

In certain embodiments, the program can cause the processor to set a standard to determine the safety level based on the first and second confidence levels. In some embodiments, safety level is determined high when the processor determines at least one of the first and second confidence levels is high. In other embodiments, safety level is determined high the processor determines both of the first and second confidence levels are low.

In some embodiments, the machine coupled with the system can be a vending machine, and the program can cause the processor to cause the vending machine fail to vend the food item when the processor determines the safety level low. In other embodiments, the machine can be a self check-out kiosk, and the program further causes the processor to cause the self check-out kiosk fail to check out the food items when the processor determines the safety level low. Still in other embodiments, the machine is a self-order machine, and the program further causes the processor to cause the self-order machine fail to processes the order of the food items when the processor determines the safety level low.

Another aspect of the disclosed subject matter includes a system for protecting a patient from adverse reaction to a food ingredient, which system is communicatively coupled with a machine. The system includes a medical database storing a patient's medical data, and a processor and a memory storing program instructions. The program instructions, when executed by the processor, cause the processor to derive a first confidence level data indicating a probability of the patient having adverse reaction to the food ingredient from the patient's medical data. In some embodiments, the patient's medical data includes patient's food sensitivity data.

The program instructions also cause the processor to obtain food ingredient information. In some embodiments, the food ingredient information can be obtained from sensor data representing a food item from a sensor device). Once the sensor data is obtained, the program instructions also cause the processor to derive food ingredient information comprising a second confidence level data indicating a probability of the food ingredient existing in the food item based on the sensor data. Then, the program instructions cause the processor to generate a safety level for the patient to consume the food item based on the first and second confidence level data, and then cause the a machine to display a food recommendation according to the generated safety level.

In some embodiments, the recommendation can include alternative food items to the food item if the second confidence value is higher than the first confidence value. In these embodiments, that the program may further causes the processor to cause the machine to display a promotional material with the alternative food items.

Another aspect of the disclosed subject matter includes a method for protecting a patient from adverse reaction to a food ingredient. The method begins with a step of deriving a first confidence level data indicating a probability of the patient having adverse reaction to the food ingredient from the patient's medical data. Then, the method continues by obtaining food ingredient information. In some embodiments, the food ingredient information can be obtained from sensor data representing a food item from a sensor device. Once the sensor data is obtained, food ingredient information comprising a second confidence level data can be derived from the sensor data. The food ingredient information indicates a probability of the food ingredient existing in the food item. Then, based on the first and second confidence level data, a safety level for the patient to consume the food item can be generated. Based on the safety level, a machine can restrict a user from an access of the food item.

Still another aspect of the disclosed subject matter includes a method for protecting a patient from adverse reaction to a food ingredient. The method begins with a step of deriving a first confidence level data indicating a probability of the patient having adverse reaction to the food ingredient from the patient's medical data. Then, the method continues by obtaining food ingredient information. In some embodiments, the food ingredient information can be obtained from sensor data representing a food item from a sensor device. Once the sensor data is obtained, food ingredient information comprising a second confidence level data can be derived from the sensor data. The food ingredient information indicates a probability of the food ingredient existing in the food item. Then, based on the first and second confidence level data, a safety level for the patient to consume the food item can be generated. Based on the safety level, a machine can display a food recommendation.

Still another aspect of the disclosed subject matter includes a computer-readable non-transitory storage medium comprising programming instructions. Then programming instructions cause the one or more processors to perform steps of operations when executed by one or more processors. The program instructions cause the processor to derive a first confidence level data indicating a probability of the patient having adverse reaction to the food ingredient from the patient's medical data. In some embodiments, the patient's medical data includes patient's food sensitivity data.

The program instructions also cause the processor to obtaining food ingredient information. In some embodiments, the food ingredient information can be obtained from sensor data representing a food item from a sensor device. Once the sensor data is obtained, the program instructions also cause the processor to derive food ingredient information comprising a second confidence level data indicating a probability of the food ingredient existing in the food item based on the sensor data. Then, the program instructions cause the processor to generate a safety level for the patient to consume the food item based on the first and second confidence level data, and then cause the machine to restrict access of the food item according to the generated safety level.

Still another aspect of the disclosed subject matter includes a computer-readable non-transitory storage medium comprising programming instructions. Then programming instructions cause the one or more processors to perform steps of operations when executed by one or more processors. The program instructions cause the processor to derive a first confidence level data indicating a probability of the patient having adverse reaction to the food ingredient from the patient's medical data. In some embodiments, the patient's medical data includes patient's food sensitivity data.

The program instructions also cause the processor to obtaining food ingredient information. In some embodiments, the food ingredient information can be obtained from sensor data representing a food item from a sensor device. Once the sensor data is obtained, the program instructions also cause the processor to derive food ingredient information comprising a second confidence level data indicating a probability of the food ingredient existing in the food item based on the sensor data. Then, the program instructions cause the processor to generate a safety level for the patient to consume the food item based on the first and second confidence level data, and then cause the machine to display recommendations according to the generated safety level.

Various objects, features, aspects and advantages of the disclosed subject matter will become more apparent from the following detailed description of embodiments, along with the accompanying drawing figures in which like numerals represent like components.

### BRIEF DESCRIPTION OF THE DRAWINGS

FIG. 1 illustrates one embodiment of food access control environment.

FIG. 2 illustrates an exemplary embodiment of the food access control environment in the shopping environment.

FIG. 3 illustrates a flowchart for one embodiment of methods for protecting patient from adverse reaction to a food ingredient.

### DETAILED DESCRIPTION

The disclosed subject matter provides systems, methods, and computer-readable non-transitory storage medium for protecting a patient from adverse reaction to a food ingredient based on the patient's medical data and food ingredient information derived from sensor data of food items and causing a machine to restrict a user from accessing to the food item or displaying a notification of food recommendation in the user device.

Throughout the following discussion, numerous references will be made regarding servers, services, interfaces, engines, modules, clients, peers, portals, platforms, or other systems formed from computing devices. It should be appreciated that the use of such terms is deemed to represent one or more computing devices having at least one processor (e.g., ASIC, FPGA, DSP, x86, ARM, ColdFire, GPU, multi-core processors, etc.) configured to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, a circuit-switched network, the Internet, LAN, WAN, VPN, or other type of network. The terms “configured to” and “programmed to” in the context of a processor refer to being programmed by a set of software instructions to perform a function or set of functions.

While many commercially-packaged food items display nutritional information on their packaging, such as the presence of fats, carbohydrate content, calories, and other ingredients, it is often impossible to find a complete listing of all ingredients or the particular details of a specific ingredient. A significant problem includes the presence of “sub-ingredients” that fall within a taxonomic category listed on the food packaging, but having the express identification of those sub-ingredients absent or otherwise hidden within the listed food items. For example, even if the food packaging displays information that the food item contains corn syrup as one of main ingredients, such information does not provide details such as whether the corn syrup contains specific types of wheat or whether it does not contain wheat. Similarly, some lecithin food additives contain or are derived from egg, while others do not. This lack of information is even more pronounced with precooked ready-to-eat food items, or already-prepared meals served in the restaurants, food stands, food trucks, etc. Consequently, individuals diagnosed with one or more food sensitivities against various types of food ingredients are often unable to make an informed and appropriate decision as to whether or not a particular dish or food item is suitable for his or her consumption.

Thus, one should appreciate that the disclosed food recommendation system and method provides numerous advantageous technical effects. For example, the food recommendation system and method of some embodiments enables up-to-date food sensitivity and/or food ingredient information by continuously learning different users' food sensitivity and food ingredients in different dishes by pulling (i.e., group-sourcing) real-time information from all users. Moreover, by matching or correlating confidence levels of patient test results with confidence levels for ingredient identification, the number of false positive and false negative results can be dramatically reduced. In addition, this disclosure allow for construction or configuration of a computing system or device to operate on vast quantities of digital data, beyond the capabilities of a human. The computing system or device is able to manage the digital data in a manner that could provide utility to a user of the computing system or device that the user would lack without such a tool.

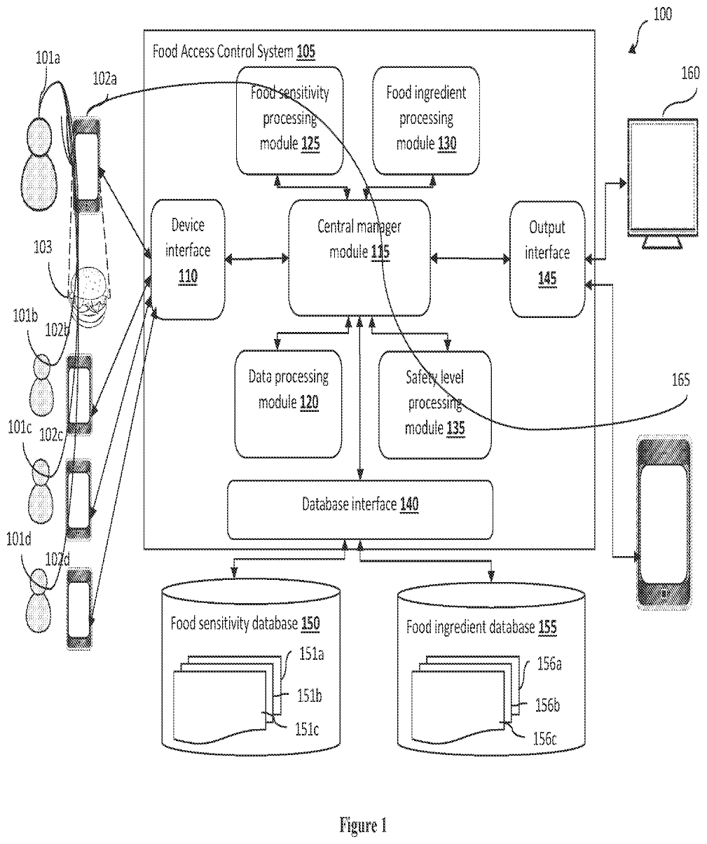

One aspect of the disclosed subject matter relates to methods, devices, and systems for controlling food access. FIG. 1 illustrates one embodiment of the food access control environment 100. In some embodiments, the food access control environment 100 includes at least one user device 102a, associated with a user 101a (e.g., patient diagnosed with a condition associated with a food sensitivity, a patient suspected to have a condition with a food sensitivity, etc.). In some embodiments, the food access control environment 100 also includes a group of individuals 101b, 101c, 101d (e.g., individuals diagnosed with a condition associated with a food sensitivity, individuals suspected to have a condition with a food sensitivity, individuals without a condition associated with a food sensitivity, etc.). In one embodiment, at least one user device 102b, 102c, 102d is associated with each individual 101b, 101c, 101d. However, it is contemplated that every individual need not be associated with a user device

In FIG. 1, the user devices 102a, 102b, 102c, 102d are illustrated as smart phones, but user devices may more generally be, for example, another type of digital devices, such as a cell phone, a smart watch, a tablet, a digital organizer, a game console, a computer, a digital camera, an appliance, a kiosk, or a biometric device, which has a memory to store data and programming instructions, and at least one processor for executing the programming instructions. In one embodiment, the user devices 102a, 102b, 102c, 102d comprise one or more sensor devices obtaining variable modalities of data. For example, the user device 102a, 102b, 102c, 102d can include optical sensors (e.g., a camera, an infrared detector, a spectrometer, etc.), chemical sensors (e.g., an electronic nose, a type of MEMS vacuum pump, etc.), or other types of sensors that are suitable to detect sound, texture, or other data modalities. In some embodiments, the user devices 102a, 102b, 102c, 102d can further include one or more location sensors (e.g., a WiFi signal strength meter, a GPS sensor, an accelerometer, etc.), which can be used to detect the location of the devices 106 or 107. As discussed further below, these sensors may provide a form of data modality (e.g., location data, etc.) that is useful in obtaining food ingredient information of a food item.

The food access control environment 100 includes a food access control system 105, which is coupled with the user devices 102a, 102b, 102c, 102d via a device interface 110. In some embodiments, the food access control system 105 is implemented on one or more computing devices having a memory that stores programming instructions, that when executed by the processor(s) of the one or more computing devices, causes the processor(s) to perform functions of the food access control system 105. As shown, in addition to the device interface 110, the food access control system 105 includes a central manager module 115, a data processing module 120, a food sensitivity processing module 125, a food ingredient processing module 130, a safety level processing module 130, an output interface 145 and a database interface 140. As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be appreciated that the structure of food access control system 105 described in FIG. 1 is illustrative, and that the structure of food access control system 105 may have a variety of different configurations. The modules shown in FIG. 1 may be reordered or even combined. For example, in some embodiments, the functions of the data processing module 120 and the functions of the food sensitivity processing module 125 may be combined, wherein functions of the food sensitivity processing module 125 are leveraged to assist in data processing. Accordingly, unless stated otherwise, any reference to a “module” should only be construed as being indicative of the function being performed by that module, and not necessarily a requirement that the particular module being referenced is separate and/or physically distinct from another module, or not coupled to another module.

The food access control environment 100 also includes a food sensitivity database 150 and a food ingredient database 155, each of which is coupled with the food access control system 105 via the database interface 140. The food sensitivity database 150 stores any data related to the food sensitivity of the patient (e.g., user 101a), including an actual food sensitivity on a food ingredient and/or a suspected food sensitivity on a food ingredient. The food sensitivity database 150 also stores any data related to the food sensitivity of one or more of the group of individuals (e.g., individuals 101b, 101c, and 101d) including their actual food sensitivities on one or more food ingredient and/or suspected food sensitivities on one or more food ingredient. For example, using the mobile device of a user, the user can report any sensitivity reaction to a food item (e.g., a dish the user consumed at a restaurant, a snack the user consumed, etc.) by sending information about the food item (e.g., an image of the food item, describe the food item, a name of the dish and the restaurant from which the user consumed the dish, etc.), and also information about the user's adverse symptoms to the food item (e.g., a type of allergic reaction, rashes, etc.). The food sensitivity data can be collected from the user, and also other individuals who use the food access control system 105. The process of collecting individuals' food sensitivity data based on the individuals' ongoing experience will be described in more detail below.

In some embodiments, the food sensitivity database 150 stores medical data of the user 101a (e.g., a patient, etc.) and/or one or more individuals 101b, 101c, 101d that are directly received from any healthcare provider (e.g., a hospital, a doctor's office, a dentist's office, a pharmacy, a lab, a doctor, a nurse, a pharmacist, an insurance provider, any point of contact for patient's healthcare, etc.). Generally, a patient's medical data generated in the doctor's office is directly transmitted to the medical database 150 with patient's consent to release the data to the database. In some embodiments, the medical database is located in a cloud server communicable with healthcare provider's and/or the patient's device via a network. In some embodiments, patient's medical data can be transmitted automatically when a health care provider places the information in his or her system (e.g., healthcare provider's computer, etc). In other embodiments, a healthcare provider can manually upload the patient's medical data in the medical database. It is also contemplated that a patient can upload his or her medical data manually to the database or the food recommendation system application.

The medical data includes any types of health-related information of the patient. For example, the medical data includes any previous personal medical history (e.g., any diagnosis received from any medical provider, etc.), any family disease history (e.g., diabetes, heart diseases, neuronal diseases, immune deficiency diseases, etc.), and any types of body check-up data (e.g., patient's blood pressure data, heart rate data, body fat data, etc.). Thus, as used herein, patient's medical data can be any data on the patient's physical or mental response associated with dietary items (e.g., food items, nutritional items, etc.) that negatively impact the patient's health.

In certain embodiments, the medical data includes the patient's food sensitivity test data. Food sensitivity test data can be obtained by any method of evaluating food sensitivity. An exemplary method of evaluating food sensitivity is described in International Patent Application Publication No. WO 2016/077808, which is incorporated herein by reference in its entirety. In another embodiment, the medical data includes a doctor's opinion or recommendation upon the patient's health condition (e.g., restricting the diet to avoid high cholesterol-containing food upon patient's high-risk cardiac condition, etc.).

In some embodiments, the food sensitivity test data can be derived from the sensitivity testing of a patient diagnosed with, or suspected of having, as disease, illness, or allergy associated with some food items or food ingredients (e.g., Irritable Bowel Syndrome, etc.) using a test kit (e.g., multi-well test plate). The food sensitivity test can be performed by contacting at least one food preparation (e.g., food item or food ingredient, etc.) with a bodily fluid (e.g., blood or saliva, etc.) of a patient that is diagnosed with or suspected of having a disease, allergy, or illness (unless stated differently, the term “disease” generally include any disease state, allergy, or illness). The bodily fluid contacts the food preparation in a condition that allows for one or more antibodies in the bodily fluid to bind to at least one component of the food preparation. Then, the amount of antibody bound to at least one component of the food preparation is measured (e.g., using immuno assay, etc) to obtain a signal. Then, the signal is compared to a reference value for the food preparation to provide a report on the patient's sensitivity for the at least one food preparation.

In some embodiments, the reference value for the food preparation will comprise an average discriminatory p-value of <0.15 for a patient group not diagnosed with or suspected of having the disease. In some embodiments, the p-value is <0.10, <0.08, <0.07, <0.06, <0.05, or even <0.025. In some embodiments, the p-value is an average discriminatory p-value determined by raw p-value, or FDR (False Discovery Rate) multiplicity adjusted p-value. In some embodiments, the antibody is selected from IgG, IgE, IgA, and/or IgM. In some embodiments, the reference value is gender stratified, meaning that p-values are determined separately based on male-only/female-only patient groups not diagnosed with or suspected of having the same disease. In some embodiments, the gender-stratified reference value for the food preparation is based on results falling within the 90th percentile value of the patient group. In some embodiments, the gender-stratified reference value for the food preparation is based on results falling within the 95th percentile value of the patient group. Diagnostic testing kits suitable for obtaining food sensitivity data include those marketed under the name InFoods® by Biomerica Inc. of Irvine, Calif.

Accordingly, in some embodiments, a patient's food sensitivity data comprises data derived from the testing of the patient for a disease using a food preparation having a reference value, wherein the reference value comprises an average discriminatory p-value of <0.15 for a patient group not diagnosed with or suspected of having the disease, allergy, or illness. In some embodiments, the food sensitivity data comprises data derived from a gender-stratified p-value. In other embodiments, the food sensitivity data comprises data derived from a disease-state-stratified p-value.

In some embodiments, the medical data includes a doctor's opinion or recommendation upon the patient's health condition (e.g., restricting the diet to avoid high cholesterol-containing food upon patient's high-risk cardiac condition, etc.). In some embodiments, the medical data received from health care providers are stored in subdirectories of the medical database 150. For example, any family disease history data can be stored in the family disease history database, the patient's blood pressure data can be stored in the blood pressure database, and the food sensitivity testing data can be stored in the food sensitivity testing database.

In addition to patient's medical data, the food sensitivity database 150 can also store medical data of one or more individuals 101b, 101c, 101d. The medical data of one or more individuals 101b, 101c, 101d includes food sensitivity test data 151a of each of one or more individuals 101b, 101c, 101d. The medical data 151a of one or more individuals 101b, 101c, 101d can also include any data related to the past and present health condition 151b (e.g., disease, genetic condition, family history, nutritional information, etc.) of the individuals 101b, 101c, 101d. Generally, it is contemplated that those data related to the past and present health condition as well as food sensitivity test data 151a of each individual 101b, 101c, 101d are considered sharable information. Thus, in some embodiments, the food sensitivity database 150 is a group-sourced database.

As used herein, group-sourcing refers to the ability of multiple persons to contribute to content. With respect to content of a group database as described by this disclosure, there may be a limited number of persons that are allowed to contribute to the content for the database or a specific subdirectories of the database (e.g., food ingredient database, food sensitivity database, etc.). However, unless otherwise stated, it should be understood reference to a “group database” or “medical database” could generally refer to data sourced on the same network. For example, a group database could be subdirectory in food sensitivity database 150, or a medical database could be a subdirectory in food sensitivity database 150.

In addition to individual's medical data, the food sensitivity database 150 can also store experience data 151c of the user 101a and/or one or more group of individuals 101b, 101c, 101d. The experience history can be directly or indirectly provided by the user 101a and/or one or more individuals 101b, 101c, 101d via the user devices 102a, 102b, 102c, 102d. For example, the user 101a or individuals 101b, 101c, 101d can enter information that they are diagnosed with IBS or suspected to have IBS, and they have experienced adverse symptoms when they consumed manila clams. For other example, the user 101a or individuals 101b, 101c, 101d can enter information that while they have not diagnosed with IBS, whenever they consumed tiger shrimp, they experienced similar symptoms with IBS.

Medical data 151a, 151b or experience data 151c provided by the user 101a and/or one or more individuals 101b, 101c, 101d via the user devices 102a, 102b, 102c, 102d can be received via device interface 110, which is communicatively coupled with the central manager module 115. The central manager module is also communicatively coupled with the data processing module 120. The medical data or experience data are processed (e.g., classified, sorted out, etc.) in the data processing module 120 are stored in the food sensitivity database 150 via a database interface 140.

The central manager module 115 of the food access control system 105 receives patients and/or other individuals' medical data 151a, 151b or experience data 151c from the food sensitivity database 150. Then, the food sensitivity processing module 125 can derive a first confidence level from the medical data received, based on one or more of a patient's diagnosis, medical recommendations or parameters provided by medical provider, a patient's experience history regarding particular food items, or the experience histories of other users. In certain embodiments, the first confidence level data comprises the patient's food sensitivity confidence level data from the patient's and/or other individuals' medical data 151a, 151b or experience data 151c. In one embodiment, the patient's food sensitivity confidence level data includes one or more confidence levels (or values) based on one or more food items or ingredients. The food sensitivity confidence level data represents a “possibility” or “probability” of a patient to exhibit a symptom (e.g., a symptom associated with an impending reaction, etc.) or an adverse reaction (e.g., allergic reactions, etc.) related to the food sensitivity when the patient consumes, or is otherwise exposed to (e.g., inhaling, touching, etc.) the food item or food ingredient. Unless stated to the contrary, as used herein, the term “food item” may comprise one or more food ingredients, while the term “food ingredient” generally refers to a single nutritional component (e.g., soybean oil, etc.) of a food item (e.g., soy sauce-flavored instant ramen, etc.).

In some embodiments, the user 101a and/or a group of individuals 101b, 101c, 101d can continually update his or her experiences with particular food items to the food access control system 105 using the user devices 102a, 102b, 102c, 102d. For example, the user 101a can provide an experience “had a headache when I tried the past food item D in restaurant E”. The patient can also add the subjective strength of the symptom (e.g., most strong, strong, moderate, weak, etc), which can be objectively converted into a sensitivity confidence value with respect to the particular food items or ingredients. This “experience history” can be stored locally in a temporary storage (e.g., memory, etc.) in the food access control system 105, and/or uploaded to (and updated on) either the food sensitivity database 150 or food ingredient database 155.

Based, at least in part, on the experience history of the user 101a with a particular food item, the food access control system 105 can determine a sensitivity confidence value for the patient on the food item. For example, if the patient experienced “very strong” symptom after consuming the food item D, then the food access control system 105 is programmed to determine that the patient is highly sensitive to food item D and assign an appropriate probability value (e.g., >90%). For another example, if the patient experienced moderate symptoms each time the patient consumed food item D, then the food access control system 105 is programmed to determine that the patient is more than likely sensitive to menu D (e.g., sensitivity confidence probability of >50%). Furthermore, the food access control system 105 of some embodiments is programmed to make different determinations based on repetition of occurrences of these adverse symptoms. For example, if the patient experienced adverse symptoms after consuming food item D only half of the time, the food access control system 105 is programmed to reduce the sensitivity confidence value with respect to that food item D for the patient. On the other hand, if the patient experienced adverse symptoms after consuming food item D every time, the food access control system 105 is programmed to increase the sensitivity confidence value with respect to that food item D for the patient.

Additionally, food access control system 105 can determine the association between the food sensitivity confidence level data and food ingredient confidence level data of the food item D. For example, if the patient's food sensitivity confidence level data (e.g., previously determined and stored in the food sensitivity database 150) indicates that the patient is highly sensitive to only two types of shellfish, and the patient experienced very strong symptom after consuming the food item D, then food access control system 105 is programmed to determine and update the food ingredient confidence level data of the food item D that the food ingredient confidence level of two types of shellfish in food item D is between 70%-100%. Accordingly, in some embodiments, the food sensitivity data and/or the food information comprises experience history data of the patient or patients.

In some embodiments, the food access control system 105 is programmed to receive food sensitivity data from others (e.g., other patients having food sensitivities, etc.) and generate the food sensitivity confidence level data of the user 101a at least partly based on the experience histories of other individuals. In these embodiments, the food sensitivity database 150 and food ingredient database 155 are configured to store food sensitivity confidence level data and food ingredient confidence level data of a plurality of individuals having food sensitivities against various types of food items. In addition, the databases 150 and 155 are also configured to store other individuals' experience histories on food items and its analysis data (e.g., updated food ingredient confidence level data based on the experience history, etc.). For example, based on another patient's experience history on food item F and its analysis that the food item F is likely to contain a specific type of cheddar cheese, and based on the instant patient's high food sensitivity confidence level against the specific type of cheddar cheese, the food access control system 105 can generate a recommendation not to consume food item F to the instant patient. It is also contemplated that, in some embodiments, the food access control system 105 can modify or update the food ingredient confidence level based on the number and/or frequency of others' sensitivity ratings. For example, when more patients provides their experience history that food item F is likely to contain a specific type of cheddar cheese, then the food ingredient confidence level that food item F is likely to contain a specific type of cheddar cheese would be increased.

In some embodiments, the experience histories of the patient or other individuals may be used to develop the food sensitivity confidence level data that is disease, sickness, or allergy specific. For example, it may be understood that a patient group exhibiting symptoms with one category of a disease or syndrome (e.g., Irritable Bowel Syndrome—C (with constipation)) may have a different level of sensitivity for some foods when compared to patient group of another category (e.g., Irritable Bowel Syndrome—D (with diarrhea)). Accordingly, in some embodiments, the food sensitivity confidence level data may be disease stratified, wherein unique first confidence level data is assigned for each category of a disease, syndrome, sickness, or allergy.

In some embodiments, the sensor of the user's device can be used to collect ambient data (e.g., location data, temperature data, time data, etc.) in addition to food data. In these embodiments, the food access control system 105 is also programmed to use the collected ambient data to derive the food sensitivity value for the patient. For example, restaurants might source their food ingredient locally, and the patient may be more sensitive to food ingredient from a certain region than others. Furthermore, the patient may also be more sensitive to a certain food ingredient at a certain hour of the day (e.g., more sensitive to caffeine in the morning, etc.). Thus, the food access control system 105 of some embodiments is programmed to use the ambient data (e.g., location data, time data, etc.) to assist in deriving the food sensitive data for the patient.

In some embodiments, the food sensitivity confidence level data can be shown in a range of 0-100%. Thus, for example, when the patient is highly allergic to pork and is certain to exhibit an adverse reaction (or at least a symptom of an impending reaction) when the patient consumes pork, the food sensitivity confidence level of the patient against pork would be 100%. In another example, when the patient has a most likely positive chance of having a symptom when the patient consumes pork, the food sensitivity confidence level of the patient against pork can be ranged at 90-99%. In still another example, when the patient more likely than not would have a chance of having a symptom when the patient consumes pork, the food sensitivity confidence level of the patient against pork can be ranged at 50-90%. When the patient has only a possibility, or a low probability, of having a symptom or reaction when the patient consumes pork, the food sensitivity confidence level of the patient against pork can be ranged at 10-49%. When the patient has essentially no possibility of having a symptom when the patient consumes pork, the food sensitivity confidence level of the patient against pork can be 0%.

In other embodiments, instead of providing a raw value of food sensitivity confidence level, it is also contemplated that the food sensitivity confidence level data for ingredients can be represented as a high-mid-low possibility level of sensitivity. For example, if the food sensitivity confidence level is higher than a high threshold level (e.g., 75%, 80%, 85%, 90%, etc.), then the food sensitivity confidence level data for the ingredient can be represented “high”. In this example, if the food sensitivity confidence level is higher than a low threshold (e.g., 50%, 45%, 40%, 35%, etc.) but lower than the high threshold, then the food sensitivity confidence level data for the ingredient can be represented “mid”. Also, if the food sensitivity confidence level is lower than the low threshold, then the food sensitivity confidence level data for the ingredient can be represented “low”. The high and low threshold can be determined depending on the severity of general symptoms against the specific food ingredient or the individual's biographical information (e.g., age, gender, ethnicity, medical history, etc.).

In some other embodiments, the food sensitivity confidence level data is visually displayed to the user 101a. For example, the food sensitivity confidence level data can be shown as a graphical continuous progress bar. The progress bar can be accompanied with different color representation of bars depending on the level of sensitivity (e.g., red for high level of sensitivity, yellow for mid-level of sensitivity, green for low-level sensitivity, etc.). Unless the context dictates the contrary, ranges set forth herein should be interpreted as being inclusive of their endpoints and open-ended ranges should be interpreted to include only commercially practical values. The recitation of ranges of values herein is merely intended to serve as a shorthand method of referring individually to each separate value falling within the range. Unless otherwise indicated herein, each individual value within a range is incorporated into the specification as if it were individually recited herein. Similarly, all lists of values should be considered as inclusive of intermediate values unless the context indicates the contrary.

The food access control system 105 is also coupled with a food ingredient database 155. The food ingredient database 155 includes food ingredient data comprising any types of information related to the food ingredients of many different food items. For example, the food ingredient data can include pre-existing data that has been compiled with respect to the ingredients that make up food items such as ingredient and nutritional information of pre-packed, factory-manufactured food items 156a that are provided by the manufacturer. In another example, the food ingredient data can include a product identifier that is a representation (e.g., digital representation) of a food item or an object (e.g., bar code, smart code, etc.) associated with the food item. In some embodiments, the food ingredient database 155 can include ingredient information of the food items (e.g., home-cooked meals, restaurant foods, etc.) that is obtained or received from the user 101a. The food ingredient database 155 can also include ingredient information of the food items (e.g., home-cooked meals, restaurant foods, etc.) that is obtained or received from the user 101a and/or the group of individuals 101b, 101c, 101d.

In some embodiments, the digital representation of the food item may already be pre-loaded on the food access control system 105, making it accessible for selection at a later time by the patient, user, or any group of individuals through a scrolling function on device interface 110. These digital representations may be input into the food access control system 105 at any time by the user or any individual maintaining the system. For example, the user 101a may independently upload a digital representation acquired from a third party into the food access control system 105 through device interface 110. In another example, the user 101a may directly add food ingredient information to the food access control system 105 through device interface 110 and store it in the food ingredient database 155, and the food access control system 105 can obtain the food ingredient information if necessary.

It is contemplated that a copy of at least one of food sensitivity database 150 and food ingredient database 155 can be stored in one of the user devices 102a, 102b, 102c, 102d. In this embodiment, it is preferred that the food access control system 105 is also executed in that one user device. It is also preferred that the copy of the food sensitivity database 150 and/or food ingredient database 155 stored at the user device is synchronized with other copies of food sensitivity database 150 and/or food ingredient database 155 periodically (e.g., every hour, every day, etc.). It is also contemplated that at least one of food sensitivity data base 150 and food ingredient database 155 can be located in a third party's computer and be accessible by the user device via a network.

In some embodiments, one or more of the user devices 102a, 102b, 102c, 102d can receive or obtain sensor data, such as a digital representation of an object associated with a food item 103. The sensor data may comprise one or more of data modalities (e.g., image data, time data, text data, ambient data, etc.), which can be used as a product identifier. In one embodiment, the object can be a food menu (either in text format or in combination of a graphic and a text formats), a photograph of a food item itself (e.g., a cooked dish, a chunk of meat, mixed vegetables, a bottle of juice, a glass of wine, etc.), a photograph of a food packaging or any types of identifiers that can be associated with or represent the food item (e.g., a bar code, a smart code, a food symbol, etc.). For example, the object can be a mark, a logo, or a symbol including those found on food packaging or found at restaurant (e.g., restaurant's trademark, trade dress, menus, information charts, etc.). In other embodiments, the object can be comprises physical or chemical attributes associated with the food item (e.g., spectral data, chemosensing data, etc.).

Once the digital representation (e.g., image data, spectral data, chemosensing data) of the object is obtained or received, the food ingredient processing module 130 of the food access control system 105 can extract/derive food information based on the digital representation. The food information can include food ingredient information, food nutritional information, or cooking method information. For example, from image data of a chop salad from a franchise restaurant A, the food ingredient processing module 130 may detect various ingredients based on shapes, colors, and textures of food ingredients that can be included in the chop salad. For another example, from the same image data of the chop salad from a franchise restaurant A, the food ingredient processing module 130 may obtain the cooking recipe of the chop salad and/or nutritional information (e.g., calories, fat content, etc.) from the franchise restaurant. In some embodiments, food information can be uploaded to and stored in the food ingredient database 155. In other embodiments, food information can be stored in a third party's database.

The image data of an object associated with a food item can be extracted by food ingredient processing module 130 according to methods known to those skilled in the art. In some embodiments, food ingredient processing module 130 is adapted to carry out optical character recognition (OCR) of the digital representation to extract image data associated with any codes, text, shapes, or symbols contained therein. In some embodiments, food ingredient processing module 130 may comprise other functionalities such as edge detection, cropping, color balancing, contrast enhancement, spatial filters, noise reduction filters, image analysis algorithms, framegrabbing, or deskewing, which can all be used to provide image data that can provide food information about the food item.

Applicant has surprisingly discovered, in some embodiments, that food information can be effectively and accurately determined from an object without the need to utilize template-based matching of the image data, or other methods of redundancy analyses to match the image data with pre-loaded templates saved on the system. In some embodiments, the user can leverage the ease of capturing a bar code, smart code, or other symbols, which can be used by the system to accurately identify the food item and match it with the food ingredients associated with the food item/bar code stored in the system.

In some embodiments, the digital representation may provide a level of image data that is not complete enough for the recommendation engine to determine the appropriate level of food information. For example, in some embodiments, the image data may comprise extracted data regarding the shape and color of a fruit or vegetable, but the food access control system 105 still cannot correlate the image data with food ingredient data stored on the food ingredient database 155. Accordingly, in some embodiments, the food access control system 105 may prompt the user 101a or the group of individuals 101b, 101c, 101d to provide a positive identification of the food item by making the appropriate selection via device interface 110 from truncated list of fruits and vegetables. Once selected, the food ingredient processing module 130 will be able to make the appropriate correlation with food ingredient data stored in the food ingredient database 155.

In some embodiments, receiving food information about the food item may comprise any number of known or emerging technologies capable of helping to provide pertinent food ingredient data. For example, in some embodiments receiving food information about the food item may comprise spectral analysis or chemosensing of the food item.

In some embodiments, spectral analysis of the food item may comprise exposing the food item to electromagnetic radiation, and detecting incoming electromagnetic radiation emitted by the food item. This may be accomplished by a device designed specifically to determine the food ingredient contents of a food item by exposing it to some forms of radiation, such as a laser or spectrum-narrowed LED. Exemplary spectral devices include, but are not limited to, those described in U.S. Pat. No. 9,212,996, which is incorporated herein by reference in its entirety. In some embodiments, the target food item will absorb some wavelengths of light depending on its composition, and emit unabsorbed light back to the device. In some embodiments, the device will further comprise a grating and/or spectrograph capable of separating the incoming electromagnetic radiation into a frequency spectrum. This process may be aided by the use of a lens, which can help diffract and separate the incoming radiation. The frequency spectrum may then be emitted into a detector, which is then capable of producing an electrical or electronic signal, which, under the control of controller integrated circuit, can be digitized and transmitted in packets over the network or bus connection by network microcontroller. In some embodiments, the digitized spectrum then undergoes processing on a network subsystem(s), which is capable of analyzing the data and providing food information through a ranking process based on the data received. Thus, in some embodiments, the method comprises converting the electromagnetic radiation emitted from the food item into digital food component data, and correlating the digital food component data with food ingredient data using a data processing module. Like other embodiments described herein, the food ingredient data can be preexisting and stored on the network for accessing when needed. In this particular embodiment, the food ingredient data can comprise previously-compiled spectral data for particular food ingredients, which can be matched or correlated with electromagnetic data obtained from the target food item. In some embodiments, the spectral device can be integrated into a mobile device or tablet capable of processing the data through a software engine housed on the device, and/or by connection to a network with access to the relevant software engines and databases.

In some embodiments, chemosensing of the food item comprises exposing the food item to a chemosensor device, and detecting an incoming chemical signal emitted by the food item. In some embodiments, chemosensing may comprise an “electronic nose” or “machine olfaction” or “artificial olfactometry,” in which volatile (e.g., gaseous) chemical signatures of a target food item are detected and analyzed for composition. In some embodiments, the method comprises converting a detected chemical signal from a food item into digital food component data, and correlating the digital food component data with food ingredient data using a data processing module. In some embodiments, the food ingredient data can be preexisting and stored on the network for accessing when needed. In this particular embodiment, the food ingredient data can comprise previously-compiled chemical signatures for particular food ingredients, which can be matched or correlated with chemical signal data obtained from the target food item. In some embodiments, the chemosensor device can be integrated into a mobile device or tablet capable of processing the data through a software engine housed on the device, and/or by connection to a network with access to the relevant software engines and databases.

As mentioned above, the sensor of the user's device can be used to collect ambient data (e.g., location data, temperature data, time data, etc.) in addition to food data. In these embodiments, food ingredient processing module 130 is also programmed to use the collected ambient data to derive the food ingredient value for a food item. For example, restaurants might source their food ingredient locally, and the same dish even from the same restaurant chain but from different restaurant locations can include slightly different food ingredients. Additionally, temperature might cause some food item to undergo chemical reaction to produce different ingredients than the ones exist when the food item is fresh. Thus, the food ingredient processing module 130 of some embodiments is programmed to use the ambient data (e.g., location data, temperature data, time data, etc.) to assist in deriving the food ingredient value for a food item.

In another embodiment, the food ingredient information can be represented second confidence level data. In certain embodiments, the second confidence level data comprises food ingredient confidence level data. Food ingredient confidence level data represents a possibility or probability that the food item of the sensor data contains a specific ingredient(s). Thus, the food ingredient confidence level is derived by identifying a food ingredient, and then assigning the possibility or probability that is likely to exist in the food item.

In some embodiments, the at least one ingredient confidence value may be independently calculated or compiled by input from physicians and patients through their experience history. For example, a patient that only has a known sensitivity to gluten that exhibited a very mild adverse reaction to eating a Brand X cookies could upload that experience to the food ingredient database 155, which may provide the basis for an assigned ingredient confidence value in food ingredient database 155 reflecting a 50% probability that Brand X cookies contain gluten. Similarly, a physician examining the ingredients of Brand Y sushi could note the presence of “imitation grab meat” in the ingredient listing, which may provide the basis for an assigned ingredient confidence value in the food ingredient database 155 reflecting a 10% probability or less that Brand Y sushi actually contains real crabmeat.

In yet another embodiment, detailed information regarding the confidence value of ingredients in some food items can be obtained from the manufacturer or provider of the food item, and made available to the food recommendation system to directly receive food information with an accurate ingredient confidence value. For example, in some embodiments, a restaurant can provide food item and food ingredient data associated with some menus in the food ingredient database 155, which may be updated periodically to maintain accuracy. In this manner, the second confidence level can be maintained at an extremely accurate level, whereby “educated guesses” based on any incomplete or inaccurate image data does not need to be relied upon by the engine.

In some embodiments, the user 101a, the group of individuals 101b, 101c, 101d, or any other third party can manually input the identity of the food item and/or food ingredients into the food access control system 105 by using the device interface 110. This food ingredient group data can then be utilized by the food ingredient processing module 130 by correlating it to confidence level data about the food item and/or its ingredients stored on the food ingredient database 155. In another embodiment, as discussed above, the identity of the food item and/or its ingredients can be determined by food ingredient processing module 130 and any image data it extracts from a digital representation of on object associated with the food item, which can similarly be correlated to pertinent information related to the relevant food ingredients of the food item and any confidence level data associated therewith.

The food ingredient confidence level data can be shown in a range of 0-100%, which represents the “possibility” or “probability” that the food item contains a specific food ingredient. For example, when the food packaging displays that the food item contains pork, then the food ingredient confidence level can be 100%. Yet, when the chop salad contains a plurality of pieces of ham, and the chance that the ham is a pork ham rather than a turkey ham is 60:40, then food ingredient confidence level of pork can be 60%. For another example, if the menu of a restaurant B represents that the menu C is a vegan menu, then the food ingredient confidence level of pork can be closed to 0%.

In other embodiments, instead of providing a raw value of food sensitivity confidence level, it is also contemplated that the food sensitivity confidence level data for ingredients can be represented as a high-mid-low possibility level of containing specific ingredient. For example, if the food ingredient confidence level is higher than a high threshold level (e.g., 75%, 80%, 85%, 90%, etc.), then the food ingredient confidence level data for the food item can be represented high. In this example, if the food ingredient confidence level is higher than a low threshold (e.g., 50%, 45%, 40%, 35%, etc.) but lower than the high threshold, then the food sensitivity confidence level data for the food item can be represented mid. Also, if the food ingredient confidence level is lower than the low threshold, then the food ingredient confidence level data for the food item can be represented low. The high and low threshold can be determined depending on many variables (e.g., cooking methods, amount of the ingredient expected to be in the food item, etc.).

In some other embodiments, the food ingredient confidence level data is visually displayed to the user 101a or other users. For example, the food ingredient confidence level data can be shown as a graphical continuous progress bar. The progress bar can be accompanied with different color representation of bars depending on the level of sensitivity (e.g., red for high level of possibility, yellow for mid-level of possibility, etc.).

Unless otherwise stated, it should be understood that the elements of the methods described herein can generally occur in any particular order that still allows for the generation of a reliable food recommendation. Thus, for example, in some embodiments it may not be necessary to receive food sensitivity data before receiving food information about a food item. In fact, in some embodiments, it may be desirable to first receive food information about a food item in order to determine the confidence level of some food ingredients being present in a food item. In this embodiment, the system could then receive any food sensitivity data of the patient as it specifically relates to those relevant food ingredients, without having to access data related to any and all food ingredient sensitivities generally associated with the patient.

In one embodiment, the food access control system 105 can perform machine learning with respect to determining the food sensitivity confidence level. The food sensitivity test data and/or experience data of the user 101a, or a group of individuals 101b, 101c, 101d. In this embodiment, the food access control system 105 can identify a pattern of food sensitivity data or food ingredient data by the user 101a and/or a group of individuals 101b, 101c, 101d. For example, when the food access control system 105 receives increasing number of food sensitivity data from the suspected IBS patients group that more patients have had adverse reaction to the Alaska king crab caught last winter season. Then, the food access control system 105 can identify the pattern that suspected IBS patients group now have increased sensitivity to the Alaska king crab caught last winter season.

Once the pattern is identified, then the food access control system 105 can correlate the pattern with a probability or possibility of the patient to have an adverse reaction to the specific food ingredient. For example, from the pattern that suspected IBS patients group now have increased sensitivity to the Alaska king crab caught last winter season, the food access control system 105 can correlate that IBS patients or a person who has similar symptoms with IBS patients has a higher probability or possibility to have an adverse reaction to the Alaska king crab caught last winter season. Then, the food access control system 105 can automatically update the food sensitivity confidence level for the user (the patient 101a) that the food sensitivity confidence level for the Alaska king crab caught last winter season increases.

Similarly, the food access control system 105 can perform machine learning with respect to determining the food ingredient confidence level. In this embodiment, when the food access control system 105 receives more data from a group of people with high sensitivity to tiger shrimps that many of them had an adverse reaction to the fishcake soup in Restaurant A. Then, the food access control system 105 automatically updates the food ingredient confidence level for the fishcake soup in Restaurant A.

Once the food sensitivity confidence level data and food ingredient confidence level data is generated, the safety level processing module 135 is configured to compare the food sensitivity confidence level data and food ingredient confidence level data and generate a safety level for the user 101a to consume the food item. In some embodiments, the safety level is calculated by considering the food sensitivity confidence level and the food ingredient confidence level equally. However, it is also contemplated that the safety level is calculated by weighing the food sensitivity confidence level more than the food ingredient confidence level or vice versa.

In some embodiments, the safety level can be represented in a range of 0-100%. For example, when it is determined that absolutely safe for the user 101a to consume a food item 103 (e.g., the patient has no food sensitivity to all food ingredients in the food item 103 or the food item 103 has no possibility to include any food ingredients that the patient may have adverse reaction to, etc.), then the safety level can be 100%. In contrast, when it is determined that absolutely dangerous for the user 101a to consume a food item 103 (e.g., the patient has severe food sensitivity to at least one or more food ingredients in the food item 103 or the food item 103 has 100% possibility to include any food ingredients that the patient may have adverse reaction to, etc.), then the safety level can be 0%.

For another example, if the food sensitivity confidence level data is lower than food ingredient confidence level data, and the food sensitivity confidence level data is higher than 50%, then a safety level of less than 40% can be generated. For another example, if the food sensitivity confidence level data is higher than food ingredient confidence level data and the food sensitivity confidence level data is close to 100%, then a the safety level of over 90% can also be generated. Yet, if the food sensitivity confidence level data is higher than food ingredient confidence level data, and the food sensitivity confidence level data is close to 50%, then a safety level of 50% can be generated. If the food sensitivity confidence level data and food ingredient confidence level data are both lower than 50%, then safety level of lower than 25% can be generated.

In other embodiments, instead of providing a raw value of safety level, it is also contemplated that the safety level data for the user 101a to consume the food item 103 can be represented as a high-mid-low possibility level. For example, if the safety level is higher than a high threshold level (e.g., 75%, 80%, 85%, 90%, etc.), then the safety level data for the food item can be represented high. In this example, if the safety level is higher than a low threshold (e.g., 50%, 45%, 40%, 35%, etc.) but lower than the high threshold, then safety level data for the food item can be represented mid. Also, if the safety level is lower than the low threshold, then the safety level data for the food item can be represented low. The high and low threshold can be determined depending on many variables that are considered to determine the food sensitivity confidence level and food ingredient confidence level.

In some other embodiments, the safety level data is visually displayed to the user 101a or other users. For example, the safety level data can be shown as a graphical continuous progress bar. The progress bar can be accompanied with different color representation of bars (e.g., red for low safety level, yellow for safety level, green for high safety level, etc.).

Once the safety level data is generated by the safety level processing module 135, the safety level processing module 135 can also generate recommendation on the food item based on the safety level. The recommendation generated in the safety level processing module 135 can be displayed in a display device 165 via the output interface 145. In certain embodiments, display device 165 is user device 102a. The safety level processing module 135 is configured to create or cause the display device 165 to create text data, sound data, or graphic data corresponding to each notification type (e.g., “High Warning”, “Moderate Warning”, or “No Warning”). In some embodiments, the notification is displayed with at least one of text data, sound data, or graphic data. In other embodiments, the notification can be displayed with a combination of two or more types of data (e.g., text warning with an alarm sound, a text warning with a graphic warning sign, a graphic warning sign with an alarm sound, etc.). In other embodiments, the safety level processing module 135 is configured to create the ranking among multiple recommendations (e.g., high warning goes first and no warning goes last, etc.), and is also configured to create the notification based on the ranking.

The safety level processing module 135 can also generate recommendations of alternative food items that may be of a similar food category, but have no warning or a low warning if a High or Moderate warning is displayed with the original food item target. In some embodiments, alternative food item(s) can be displayed with the original food item warning notification, or as a separate link associated with the warning notification. In some embodiments, the alternative food item can be displayed with the lowest ranking as an alternative to the high or moderate ranking.

In certain embodiments, the user 101a is associated with the display device 165. However, it is also contemplated that the recommendation can be displayed in the display device 165 is not directly associated with the patient (e.g., a caregiver's device, a cook's device, etc.).

In some embodiments, the food recommendation or alternative food recommendation may be associated with promotional material. The promotional material may comprise sponsored, third-party advertisements for foods and/or services associated with the food recommendation. In some embodiments, the promotional material comprises at least one of a brand advertisement, a product rebate, a product coupon, or a product instant savings notice (e.g., $1.00 off credited at supermarket checkout, etc.).

In some embodiments, the food sensitivity confidence level can comprise any significance level of dietary ingredient to the patient's health condition. Thus, in some embodiments, the food access control system 105 is configured to receive diagnosis results or recommendations from medical providers to generate food recommendations to the user (e.g., patients 101a, etc). For example, the patient's medical data in the food sensitivity database 150 may comprise a doctor's recommendation to restrict high-cholesterol containing foods for improving or maintaining the patient's cardiac condition. Then, the food access control system 105 can compare the food ingredient confidence level of food items (e.g., cholesterol types or amounts in the food items) and provide recommendation to the user 101a or other users (e.g., caregivers, etc.).

In some embodiments, the food access control system 105 is further configured to obtain medical data associated with a plurality of persons (e.g., family of four persons, etc.) to provide a plurality of recommendations or to provide a best suitable recommendation meeting all persons' food sensitivity confidence level data. For example, a family of four persons can have multiple health conditions or food sensitivities. The food recommendation engine 110 is configured to obtain all medical data of the family members, and can provide multiple recommendations at the same time (e.g., avoid menu A, C, and D in restaurant F), or the best suitable recommendation for the entire family (e.g., the best menu that you can all share is menu G in restaurant F).

The food access control system 105 can also modulate the operation of a machine 160 that is communicatively coupled with the food access control system 105 according to the safety level data generated in the safety level processing module 135. In some embodiments, the machine can be at least one of the user devices 102a, 102b, 102c, 102d. In other embodiments, the machine can be different devices other than the user devices 102a, 102b, 102c, 102d, which can be communicatively coupled to the at least one or more user devices 102a, 102b, 102c, 102d. For example, the machine 160 can be a vending machine with various food items. The food access control system 105 can configure the vending machine not to vend a food item that has low safety level to a person who attempts to use the vending machine to purchase the food item. For another example, the machine 160 can be a self-order kiosk for food items. The food access control system 105 can configure the self-order kiosk not to display the food ingredient (e.g., mustard sauce for a hamburger, etc.) from the menu list, or prevent from checking out the order if the ordered food item contains food ingredient with low safety level. One of the self-order kiosks includes an online shopping or online order system (e.g., webpage, a mobile application, etc.) that displays warning signs or refuses to check out the user's orders when the user attempts to place, or places a food item with low safety level in the shopping cart of the online order system. In this scenario, the computer can label or mark the order of the food item in the shopping cart in a specific color (e.g., red text or highlighted, etc.), or attach a warning sign (“!” mark or “WARNING”, “DANGER”, etc.), or even refuse to place the food item in the shopping cart.

For still another example, the machine is a beverage-making machine (e.g., capsule coffee/tea machine, etc.), and the food access control system 105 can configure the beverage-making machine not to brew the beverage and/or providing a warning sign on the LED screen of the machine, if the beverage is likely to contain food ingredient (e.g., caffeine, etc.) with low safety level. For still another example, the machine is a robot cook that receives an order for dishes from the patients. Based on the health condition that requires food restriction as well as safety levels of food items, the food access control system 105 can configure the robot cook to refuse to cook a specific dish, provide verbal or text recommendation to the user 101a for alternative dishes, or cook the ordered dish with or without content of the user 101a using alternative ingredient that has higher safety level for the patient to consume.

It is contemplated that the machine 160 can also be any computer that provides browsing functions and/or location-detection functions. For example, the machine 160 can be a computer, and the food access control system 105 can configure the computer to limit the user access to some specific food items by providing additional filtering in the browser (e.g., not showing specific food items in the on-line ordering system, not showing some recipes using the specific food items).

For another example, the machine 160 is a computer coupled with a refrigerator or a pantry that the food access control system 105 can configure the computer to automatically cancel the order of a food item for restocking the refrigerator or a pantry if the food item has a low safety level to the user of the refrigerator or the pantry. Instead, the food access control system 105 can configure the computer to automatically order a substitute (or recommended alternative) food item when the user of the refrigerator or the pantry attempts to order the food item with a low safety level.

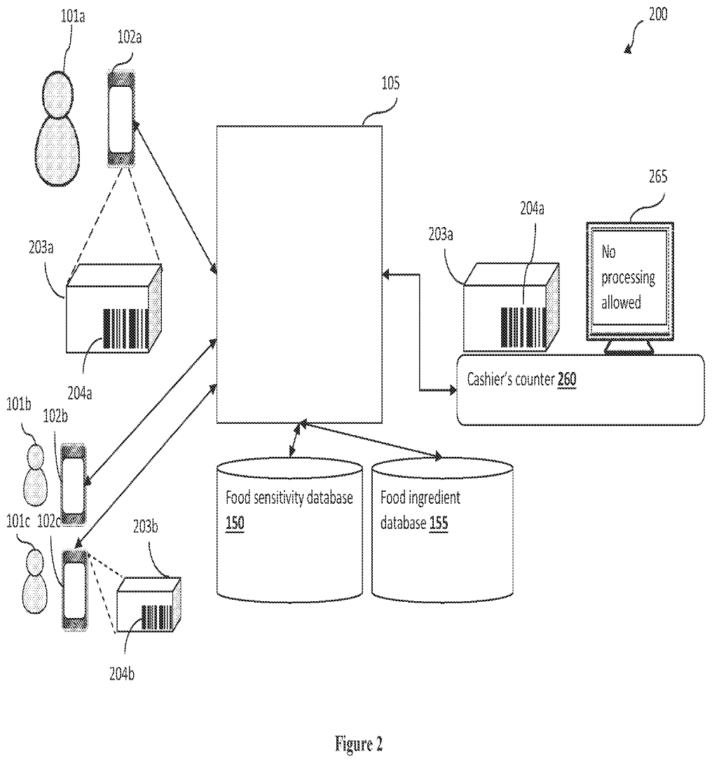

FIG. 2 shows one exemplary food access control environment 200. In this food access control environment 200, the food access control system 105 is communicatively coupled with the user 101a, and other individuals 101b, 101c, who are coupled with user devices 102a, 102b, 102c, respectively. The user 101a is in the supermarket and considers purchasing a boxed salad 203a for her lunch. The user 101a recently diagnosed with a mild IBS and have had adverse reactions (e.g., diarrhea, etc.) whenever she had grilled Italian zucchini. Her medical information of mild IBS diagnosis is transmitted from her medical data storage at her doctor's office stored in the food sensitivity database 150 after being processed at the food access control system 105. The patient's experience information about grilled Italian zucchini is entered by the patient and also stored in the food sensitivity database 150 after the data is processed at the food access control system 105. Food sensitivity database 150 also stores food sensitivity data of another individual 101b who also have diagnosed with a mild IBS with similar symptoms with the patient 101a. The food sensitivity data of another individual 101b indicates that the individual 101b have had adverse reaction to peanut oils. Based on the patient's medical data and experience data, as well as other individual's (with the same disease and similar symptoms), the food access control system 105 determines that the food sensitivity confidence level of the Italian zucchini is high (e.g., more than 90%, etc.), and the food sensitivity confidence level of the peanut oil is likely high (e.g., more than 70%, etc.).

When the user 101a is interested in purchasing the boxed salad 203a having a barcode 204a, the patient takes a photograph of the content of the boxed salad 203a and/or scans the barcode on the exterior of the salad box with the camera of the patient's user device 102a. The visual representation of the salad content and/or the barcode is transmitted to the food access control system 105, where the food ingredient information is extracted based on the visual representation. For example, from the photograph of the content of the boxed salad, the data processing module 120 of the food access control system 105 can extract the information that the boxed salad may contain lettuces, carrots, cheddar cheese, Brussels sprouts, and Italian zucchini based on the shape and color of the content. For other example, from scanned barcode 204a, the data processing module 120 of the food access control system 105 can extract the information that the boxed salad 203a that in addition to lettuces, carrots, cheddar cheese, Brussels sprouts, and Italian zucchini, the boxed salad 203a contains crushed walnuts and minced raisins. Based on the information, the food access control system 105 can determine the food ingredient confidence level of the boxed salad 203a that the food ingredient confidence level for lettuces, carrots, cheddar cheese, Brussels sprouts, Italian zucchini, crushed walnuts and minced raisins are high (e.g., over 90%, etc.)

The food ingredient database 155 may also include food ingredient information for the same boxed salad 203b having a barcode 204b that is generated by and transmitted from another individual 101c. The individual 101c have not diagnosed with IBS and does not have any symptom of IBS. Yet, the individual 101c have had adverse reactions on peanut oil. The individual 101c had purchased the same boxed salad 203b having a barcode 204b, and had an adverse reaction over the salad. The individual 101c entered food ingredient information that he suspects that the boxed salad 203b may contain peanut oil. Based on the individual's 101c experience data, the food access control system 105 can update the food ingredient information of the boxed salad 203b that the food ingredient confidence level that the boxed salad 203b contains peanut oil is moderate to high (e.g., 50-75%, 60-80%, etc.)

Based on the food sensitivity confidence data and the food ingredient confidence data, the food access control system 105 can determine the safety level for the user 101a to consume the boxed salad 203a. Because the food sensitivity confidence level is high for Italian zucchini and moderate-high (likely high) for peanut oil, and the food ingredient confidence level for Italian zucchini and peanut oil is high and moderate-high, the safety level for the user 101a to consume the boxed salad 203a is likely low.

The food access control system 105 is coupled with a cashier's counter 260, which includes a display 265. Once the food access control system 105 determines that the safety level for the user 101a to consume the boxed salad 203a is low, the food access control system 105 can cause the cashier's counter 260 refuse to check out of the boxed salad 203a when the patients attempts to check out. In addition, the food access control system 105 can cause the display 265 associated with the cashier's counter to display a warning sign to the cashier (e.g., “NO PROCESSING ALLOWED”, etc.) or a warning sign to the patient (e.g., “DANGEROUS TO CONSUME”, etc.). Further, the food access control system 105 can cause display 265 to show the list of alternative food items safer for the user 101a to consume, and cause the cashier's counter 260 to print a promotional material including coupons for the alternative food items.

In certain embodiments, virtual reality or augmented reality-type programs and devices may be integrated into the systems and methods described herein. In certain embodiments the food access control system may provide user 101a with an augmented reality platform at one or more of the stages of methods described herein. For example, an augmented reality could be provided to user 101a during the steps of obtaining sensor data and/or generating a safety level. The capturing of real-time video, digital representations, and/or other sensor data could be augmented to provide user 101a with virtual options or recommendations. In such embodiments, virtual food recommendations or alternatives may be provided to user 101a on device 102a (e.g., smartphone, smartwatch, virtual reality headset, etc.), which may selected or scrolled though by the user.

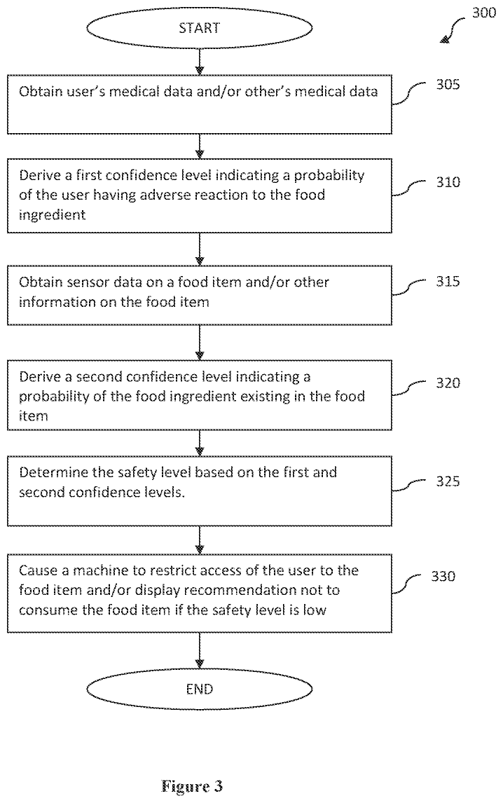

Another aspect of the disclosed subject matter relates to method for protecting a patient from adverse reaction to a food ingredient. FIG. 3 illustrate a flowchart of one embodiment of the methods. The methods begins with a step 305 of obtaining or receiving the user's (e.g., patient's) medical data and/or other individuals' (e.g., individuals having same disease or similar symptoms, etc.) medical data. In some embodiments, the medical data is directly transmitted from a medical provider or a healthcare provider (e.g., a hospital, a doctor's office, a dentist's office, a pharmacy, a lab, a doctor, a nurse, a pharmacist, an insurance provider, any point of contact for patient's healthcare, etc.) under the patient's consent to release the data. In one embodiment, the medical data includes food sensitivity test data and/or experience data of one or more individuals. Once the medical data is obtained or received, the medical data is stored in the food sensitivity database. Then, based on the medical data, the method continues with a step 310 of deriving a food sensitivity confidence level (a first confidence level), which indicates a probability of the user having an adverse reaction to the food ingredient.

The method continues with a step 315 of obtaining sensor data (e.g., a digital representation) of an object (e.g., a bar code, a photo of a cooked dish, etc), associated with a food item. In one embodiment, the image data is acquired by an image acquisition device (e.g., a camera on the mobile device). However, it is contemplated that any suitable sensor data to extract food ingredient information can be used. Also, it is contemplated that any pre-existing, pre-processed image data (e.g., screen captured image data on the personal computer, etc.) can be used.

Once the sensor data is acquired, the method further continues with a step 320 of deriving food ingredient confidence level (a second confidence level) from the sensor data. The food ingredient confidence level indicates a probability or possibility that a food ingredient exists in the food item. Then, the method continues with a step 325 of determining a safety level for the patient to consume the food item based on the food sensitivity confidence level and the food ingredient confidence level. If the safety level is low (e.g., dangerous for the patient to consume the food item, etc.), the method continues with a step 330 of causing a machine to restrict access of the user to the food item. The step 330 may also include to display recommendations to the client or the third party not to consume the food item or not to provide the food item to the patient.

It should be apparent to those skilled in the art that many more modifications besides those already described are possible without departing from the disclosed concepts herein. The disclosed subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A system for protecting a patient from adverse reaction to a food ingredient, wherein the system is communicatively coupled with a machine, the system comprising:
a memory to store program instructions; and
a processor, operatively coupled with the memory to execute the program instructions to cause the processor to:
access experience data for a user and one or more groups of individuals;
for each user among a plurality of users;
receive an indication of a food item;
access food ingredient data for the food item;
derive, from medical data and using a food sensitivity processing module, a first confidence level data indicating a probability of the patient having adverse reaction when the user is exposed to a food ingredient;
derive, using a food ingredient processing module, second confidence level data representing a probability that the food ingredient exists in the food item, wherein the program further causes the processor to: identify a pattern of the group data; correlate the pattern of the group data with a probability of the food ingredient existing in the food item; and automatically update the second confidence level data based on the pattern of the group data such that the program causes the processor to derive second confidence level data from group data comprising experience history of individuals having adverse reaction to the food item, wherein extracting the food ingredient information determines at least 8 candidate ingredients, each candidate ingredient having a probability that the candidate ingredient is in the food item;
generate, based on the first and second confidence level data, a safety level for the user to consume the food item; and
automatically cause the machine to take an action associated with the food item according to a comparison of the generated safety level to a threshold level.

2. The system of claim 1, wherein the program further causes the processor to derive first confidence level data from a testing of the patient for a disease using a food preparation having a reference value, wherein the reference value comprises an average discriminatory p-value of ≤0.15 for individuals not diagnosed with or suspected of having the same disease.

3. The system of claim 1, wherein the program further causes the processor to derive first confidence level data from a group data of individuals diagnosed of same disease with the patient, wherein the group data includes a reference value of a food preparation with an average discriminatory p-value of ≤0.15 for individuals not diagnosed with or suspected of having the same disease.

4. The system of claim 1, wherein the program further causes the processor to derive first confidence level data from a group experience data diagnosed of same disease.

5. The system of claim 1, wherein the program further causes the processor to derive the first confidence level from the patient's experience history.

6. The system of claim 3, wherein the group data comprises a plurality of sensitivity ratings associated with the food preparation.

7. The system of claim 3, the program further causes the processor to:
identify a pattern of the group data;
correlate the pattern with a probability of the patient having adverse reaction to the food ingredient; and
automatically update the first confidence level based on patterns of the group data.

8. The system of claim 1, wherein the program further causes the processor to:
obtain, from a sensor device, sensor data representing a food item; and
derive, based on the sensor data, food ingredient information comprising a second confidence level data indicating a probability of the food ingredient existing in the food item

9. The system of claim 1, wherein the program further causes the processor to derive the second confidence level data of a food ingredient from group data comprising experience history of individuals having adverse reaction to the food item.

10. The system of claim 1, wherein the program further causes the processor to:
receives medical data from at least 4 other individuals and make at least 3 recommendations based at least in part on the medical data of the 4 individuals.

11. The system of claim 1, wherein the program further causes the processor to determine the safety level low when the processor determines at least one of the first and second confidence level is high.

12. The system of claim 1, wherein the program further causes the processor to determine the safety level high when the processor determines both of the first and second confidence levels are low.

13. The system of claim 1, wherein the machine is a vending machine and the program further causes the processor to cause the vending machine to fail to vend the food item when the processor determines the safety level low.

14. The system of claim 1, wherein the machine is a self check-out kiosk, and the program further causes the processor to cause the self check-out kiosk to fail to check out the food item when the processor determines the safety level low.

15. The system of claim 1, wherein the machine is a self-order machine, and the program further causes the processor to cause the self-order machine to fail to process the order of the food item when the processor determines the safety level low.

16. The system of claim 1, wherein the machine comprises a smart phone or a smart watch.

17. The system of claim 16, wherein the program further causes the processor to cause the machine to display a notification associated with risk of an adverse reaction to, and/or a recommendation not to consume, the food item when the processor determines the safety level low.

18. The system of claim 1, wherein the machine comprises a tablet or a computer.

19. The system of claim 18, wherein the program further causes the processor to cause the machine to display a notification associated with risk of an adverse reaction to, and/or a recommendation not to consume, the food item when the processor determines the safety level low.

20. The system of claim 1, wherein the action comprises at least one of restrict access to, display a notification associated with risk of an adverse reaction to, or display a recommendation not to consume.
